(19) United States
(12) Patent Application Publication
(10) Pub. No.: US 2007/0010779 A1
(43) Pub. Date: Jan. 11, 2007

(54) Blood leak monitoring method and apparatus

(76) Inventors: David S. Utterberg, Seattle, WA (US); William J. Schnell, Libertyville, IL (US)

Correspondence Address: SEYFARTH SHAW LLP, Suite 4200, 55 East Monroe Street, Chicago, IL, 60603-5803, US

(21) Appl. No.: 11/176,912

(22) Filed: Jul. 7, 2005

(51) Int. Cl. A61M 37/00

(52) U.S. Cl. 604/4.01

(57) ABSTRACT

A method for monitoring for leaks or disconnections in an extracorporeal blood circuit, comprising the steps of operating a blood pump to circulate blood through an extracorporeal blood circuit; opening a shunt connection between the arterial and venous blood flow portions; sensing the presence of air from any leaks or disconnections within the venous blood flow portion, and taking corrective action if the presence of air is noted. The shunt connection is typically periodically but only briefly opened, to check for leaks in the typically positive pressure portion of the venous set.

### BACKGROUND OF THE INVENTION

In extracorporeal blood treatment procedures such as in hemodialysis, significant efforts must be made to monitor for leaks in the extracorporeal blood circuit. Such leaks can result in the introduction of air into the blood system and, while state of the art blood sets have air bubble traps and systems for shutting down the pump in the presence of significant air bubbles, risks remain which, although remote, can be serious and even fatal. Specifically, blood is conventionally withdrawn from the patient by a blood pump, acting to generate a suction or subatmospheric pressure in an arterial blood flow portion, which sucks blood from the patient's vascular system. This blood then passes through the pump, which is typically of peristaltic type, achieving a positive pressure. Somewhere along the line, the blood typically passes through a hemodialyzer or some other blood treatment device. Then the pressurized blood is returned to the patient via a venous blood flow portion, which extends downstream from the pump to a second connection with the patient's vascular system.

While the current technology provides bubble detectors and automatic fail-safe equipment, a significant breach in the positive pressure, venous blood flow portion generally does not cause air bubbles to enter the system. Rather, the blood flows out, and in the case of a rare separation of the blood line in the venous blood flow portion, the results can be quickly fatal. Thus the bubble detector fails to sound any alarm when there is a positive pressure leak.

Brugger et al. U.S. Pat. No. 6,572,576 provides an innovative solution to this problem with a method and apparatus for leak protection in a fluid line. Basically, flow through the sections of the arterial and venous blood flow portions that connect with the patient is reversed by a flow reversing valve. Thus, the venous blood portion no longer returns blood to the patient, but draws blood from the patient under suction (negative) pressure. Thus, any breach in the line will cause the suction of air into the system, which air can be detected by a properly positioned bubble detector. A system is provided for automatic shutoff of the pump if such is noted.

Thus, a normal, extracorporeal blood treatment procedure can take place with intermittent, repeated monitoring of the system by quick switching of the flow reversing valve, for only a brief time of seconds or less. This will occur every few minutes or less, thus reducing net flow to the patient typically by no more than ten percent. If there is a leak, it will be quickly detected by the presence of air in what is normally the venous blood flow portion. The pumping can immediately be stopped, and an alarm signal raised. This procedure may save the patient's life, while conventional, current systems can fail to detect a leak or separation in the positive pressure, venous blood flow portion.

By this present invention, protection against leaks and separations in the typically positive pressure venous blood flow portion can be monitored and protected against by a simplified system, where full flow reversal of the system is not required, and which may be performed by a simplified apparatus. In some embodiments, flow through the arterial blood flow portion may continue without flow reversal. Also, by this invention, flow through a portion of the venous blood flow portion may actually be clamped and cease for a brief period of time, typically no more than one second, which can enhance the rapidity of bubble and air detection when this intermittent process is activated.

### DESCRIPTION OF THE INVENTION

In accordance with this invention, a method is provided for monitoring of leaks or disconnections in an extracorporeal blood circuit which comprises a blood pump; an arterial blood flow portion operating at subatmospheric pressure and extending upstream from the pump to a first connection with the patient's vascular system, and a venous blood flow portion extending downstream from the pump to a second connection with the patient's vascular system. Typically, an extracorporeal blood treatment device such as a hemodialyzer is provided in the flow path. However, the circuit may also comprise hemofiltration or any other type of extracorporeal blood processing, including systems where blood is passed through a cartridge which contains activated charcoal or any other material for treatment of blood.

The method comprises the steps of:

operating the blood pump to circulate blood through the extracorporeal blood circuit;

opening a shunt connection between the arterial and venous blood flow portions;

sensing the presence of air from any leaks or disconnections within said venous blood flow portion; and

taking corrective action if the presence of said air is noted.

A shunt connection is defined as a blood flow passageway that is opened between the arterial blood flow portion and the venous blood flow portion without providing a complete reversal of flow in the arterial and venous portions that are near to the patient, as taught in Brugger et al. 6,572,576 and elsewhere. Instead, by the shunt connection of this invention, flow through the arterial blood flow portion operating at subatmospheric pressure (because it is upstream from a blood pump) continues rather normally in its original flow direction toward the blood pump, although, upon opening the shunt connection, there will be a sudden surge of blood from the pressurized, venous blood flow portion to the arterial blood flow portion, since the venous blood flow portion is downstream from pump and thus subject to higher pressure. However, apart from such a pressure surge from the venous blood flow portion, the blood pump typically continues to operate normally so that flow in the arterial blood flow portion remains normally directed toward the blood pump and is not reversed, contrary to the cited prior art.

When the shunt connection is opened, the sudden reduction of pressure in the venous blood flow portion causes a negative pressure there, which causes any air bubbles which are capable of entering the system to enter the system, and be sensed by an air sensor. Under normal flow conditions, pressure is positive in the venous blood flow portion, and the flow through any leak or opening would be that of blood flowing outwardly rather than air flowing inwardly to the system. Thus, while an air sensor will not detect a leak, separation, or other breach of the venous flow portion under positive pressure conditions, air may be detected when the shunt connection is opened, indicating the presence of a leak.

Preferably, by this method the shunt connection is briefly opened and then closed, on a repeated, periodic basis so that the extracorporeal blood circuit may operate normally for most of the time, for example in one minute increments, while the presence of air may be sensed by a sensor located to sense for such air near to the second connection.

The shunt connection may be opened and closed using only a single unclamping/clamping action, typically using a single bar clamp to release and collapse a tube that defines a single flow path shunt connection for clamping action.

If desired, while the shunt connection is opened, the venous blood flow portion may also be clamped at a position to promote blood flow, through the shunt connection, from the venous blood flow portion that is downstream of the shunt connection to the arterial blood flow portion that operates at subatmospheric pressure. This promotes flow reversal in the section of the venous blood flow portion that connects with the patient's vascular system. Thus, any breaches or leaks may be detected by drawing of air bubbles into the venous blood flow portion, where they may be sensed by a bubble detector.

Typically, blood flows from the patient through the first connection with the patient's vascular system into the arterial blood flow portion and away from the patient both in the circumstances when the shunt connection is opened, and when the shunt connection is closed, when blood is circulating through the extracorporeal blood circuit.

Preferably, a sensor is located near to the second connection, to quickly sense air if a leak or separation is present, permitting shortening of the shunt-open, sensing phase down to about a second or less, to minimize a reduction in dialysis efficiency, and also to avoid setting off pressure monitor alarms in the dialysis system, which generally require more than a second of elevating pressure to actuate under normal circumstances, with respect to the presently used dialysis systems.

During the period that the shunt is opened, the arterial blood flow portion can continue to convey blood through the first connection with the patient's vascular system and convey the blood away from the patient while the flow is being reversed in at least part of the venous blood flow portion.

The above can be accomplished by the use of an extracorporeal blood circulating device which comprises:

a blood pump;

an arterial blood flow portion extending upstream from the pump to a first connection with the patient's vascular system;

a venous blood flow portion extending downstream from the pump to a second connection with the patient's vascular system;

a shunt connection permitting direct flow between the arterial and venous blood flow portions without flowing through the pump;

a first valve controlling flow through the shunt connection; and

an optional second valve positioned to block flow through a portion of the venous blood flow portion which is upstream in normal flow from the shunt connection; and

a control unit that causes the second valve to be open when the first valve is closed, and which causes the second valve to be closed when the first valve is open.

Typically, the shunt connection is opened and closed using only a single unclamping/clamping action, contrary to the prior art, where there is a complete flow reversal in the parts of the arterial and blood flow portions nearest to the patient.

### DESCRIPTION OF THE DRAWINGS

In the drawings, FIG. 1 is a schematic view of an extracorporeal blood hemodialysis system, shown in its normal mode of operation.

FIG. 2 is a schematic view of the same system, shown in the mode of operation when checking for the presence of air in the venous blood flow portion is taking place.

FIG. 3 is a schematic drawing showing the system of FIGS. 1 and 2 being shut down, because air is detected in the venous line as the result of the process of FIG. 2.

### DESCRIPTION OF SPECIFIC EMBODIMENTS

Referring to the drawings, a hemodialysis system is disclosed in which blood is drawn from the patient 10 using a conventional fistula needle set 12 that defines a first connection with the patient's vascular system. Fistula set 12 is conventionally connected to an arterial set 14, passing through a conventional air sensor 16, which is part of a set of air sensors 16, 18, so that the presence of air leaks may be detected. Such leaks may be demonstrated by the presence of air bubbles, or by emptying of blood from the tube lumen. Arterial set 14 is upstream from a section of roller pump tubing 20, positioned in a roller pump 22. Arterial set 14 may also have other, conventional components such as a bubble trap 24, which connects with a pressure monitor 26 through tubing 27 in a conventional manner. Branch connection tubing 28 is also conventionally provided for the addition of heparin and other medications as needed.

Arterial set 14 then connects to a conventional hemodialyzer 30, which also has ports 32 for the flow of dialysis fluid through the dialyzer so that the blood typically passes through the lumens of hollow fibers, while the dialysis solution passes through exterior spaces between the hollow fibers, permitting dialysis to take place. The arterial blood flow portion comprises arterial set 14, which is upstream of pump 22, while the venous blood flow portion comprises the blood flow tubing downstream of pump 22, which is venous set 34.

As is also conventional, hemodialyzer 30 has a downstream connection to a venous set for hemodialysis 34. This set has conventional components such as another bubble trap 35, a branched, connecting pressure monitor line 38, and an added branched, connection line 40 for conventional purposes.

Venous set 34 also extends through air sensor 18, and connects with another fistula set 36 that is in connection with the vascular system of the patient. Thus, blood is withdrawn through fistula set 12 by the action of pump 22. It passes through the system including dialyzer 30, and then is returned to the patient through venous set 34 and fistula set 36.

In accordance with this invention, the arterial and venous sets 14, 34 are connected together in an H-shaped tube construction 42, which provides a shunt connection tube 44 between the two flow paths of (1) the arterial blood flow portion and set 14 and (2) the venous blood flow portion and set 34. Normally, as shown in FIG. 1, shunt tube 44 between the two sets is closed by a clamp valve member 46, which may comprise a conventional bar clamp, and which compresses the flexible tubing that defines shunt tube 44, connecting between the two arterial and venous, parallel set tube portions 14a and 34a.

Thus, conventional hemodialysis proceeds in the system when it is in the configuration of FIG. 1. It should also be added that bar clamp valve 48 is optionally present to also clamp the tubing of venous set 34, but it is open at this time.

The bar clamp valves 46, 48 may be of any desired design to accomplish flow occlusion in the flexible tubing that they address.

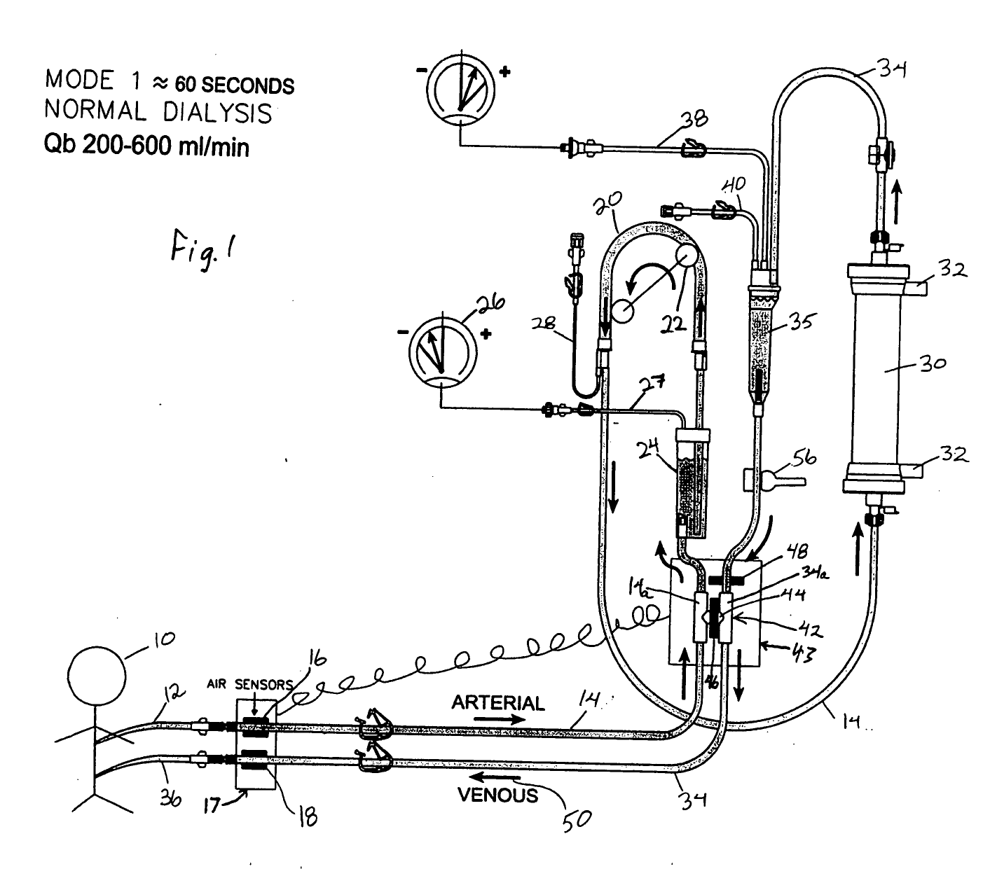

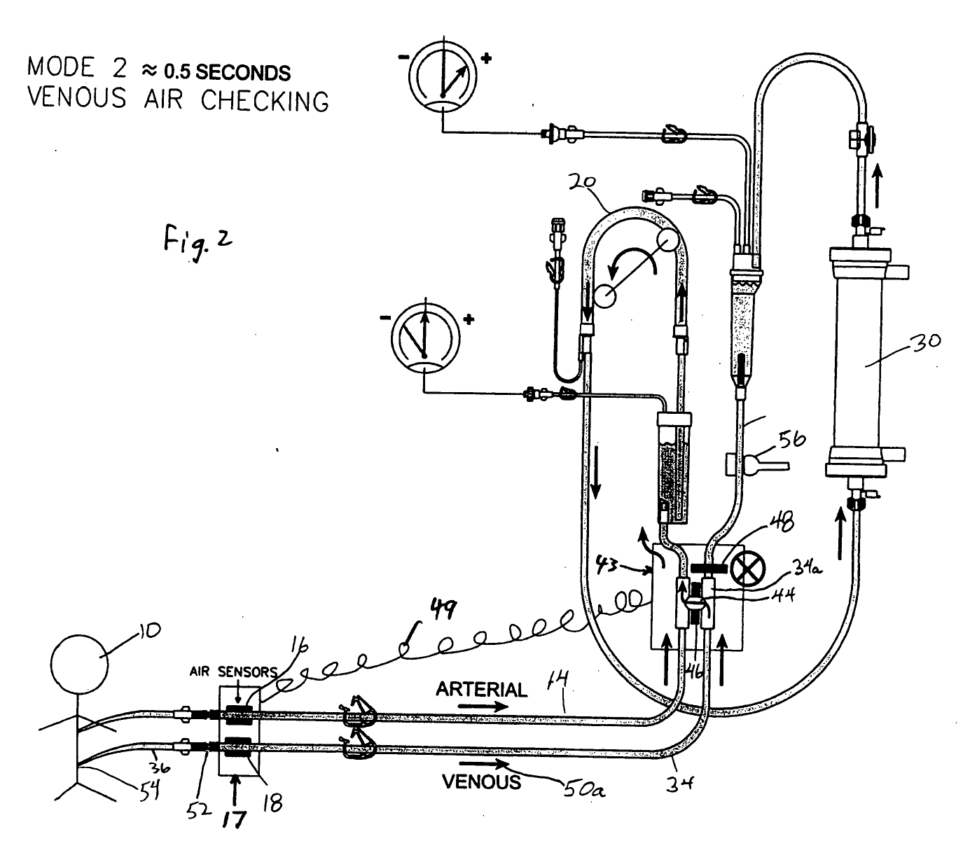

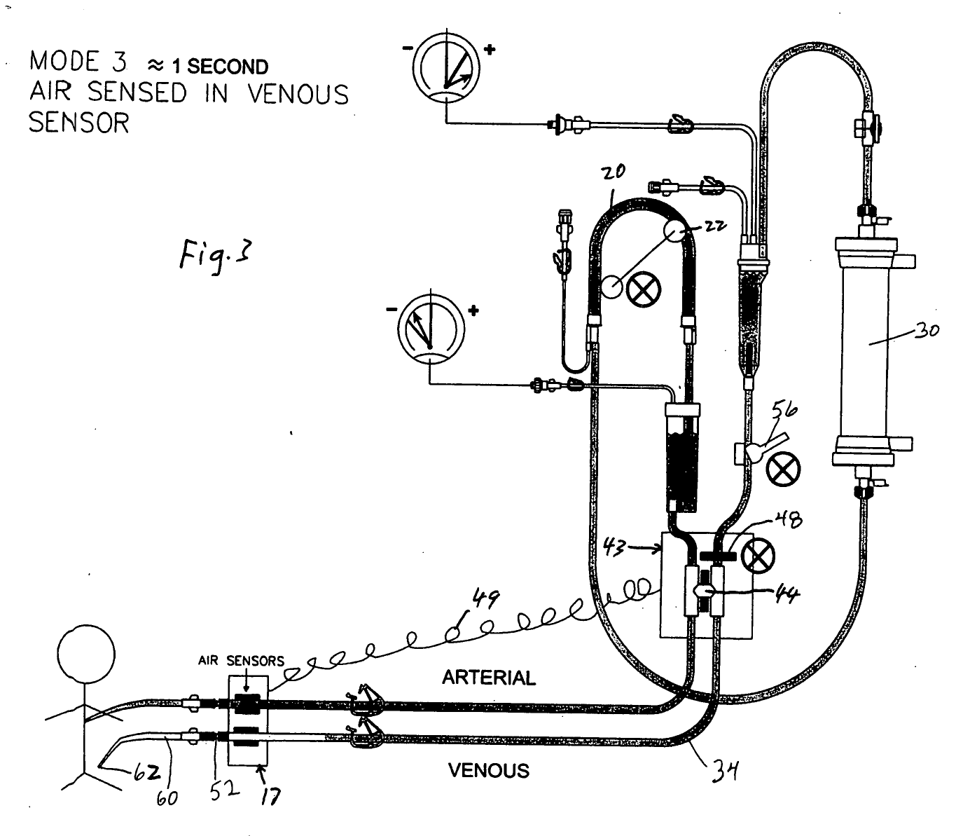

Turning to FIG. 2, the same system is disclosed, with the components of the system being identically depicted, including arterial and venous sets 14, 34, dialyzer 30, and the components that they carry.

In accordance with this invention, periodically during the dialysis procedure, for example once about every 60 seconds, bar clamp valve 46 is opened to open flow in shunt tube 44. Because the pressure in arterial tubing 14 upstream from pump tubing 20 and peristaltic pump 22 is below atmospheric by the suction action provided by pump 22, there is an immediate burst of flow through shunt tube 44 from venous line 34 to arterial line 14. The effect of this is to briefly reverse the flow in venous line 34, as indicated by the reversed flow direction of arrow 50a, compared with the direction of arrow 50 in FIG. 1. The X in a circle indicates a closed valve, in the case of FIG. 2, clamp valve 48.

Common places where a leakage or a complete separation can take place are at the junction 52 between venous set 34 and fistula needle set 36, or at the very connection of the fistula needle 54 with the bloodstream of the patient 10. Should either of these connections separate, as stated above, blood will normally flow freely out of the system without being returned to the patient, with results which, if uncorrected, will be fatal. Accordingly, the duration that a segment of normal dialysis of FIG. 1 may take place may be a function of the maximum amount of blood that a patient can afford to lose in this relatively rare accident, typically on the order of 60 seconds when flow is 200 to 600 ml./min. However, if appropriate, longer periods of time may be used, or shorter periods of time.

Thus, each session of normal dialysis as shown in FIG. 1 proceeds for a predetermined length of time, such as 60 seconds. Then, bar clamp valve 46 is raised to open flexible shunt tube 44, as in FIG. 2. It may also be desired to close bar clamp valve 48, an optional part, as indicated by the X in a circle, to block flow through a portion 34a of arterial set 34, while enhancing the reversal of flow 50a in the remainder of arterial set 34 which is closer to fistula set 36 and the patient 10 than is shunt tube 44. The resulting surge of reverse flow will bring any air that is present from the vicinity of connections 52 or 54 to air sensor 18. If air is so detected, bar clamp 48, and optionally flow valve 56, is closed long term, as indicated in FIG. 3, and an alarm may be sounded. Also pump 22 stops, as indicated by the star in a circle in FIG. 3.

Typically, the duration of the venous air checking mode of FIG. 2 may be on the order of ½ second, but of course may be greater or less as the circumstances dictate. It is desirable to keep the duration of this mode of operation to a minimum, since the most efficient dialysis may not be taking place during the operation of the venous air checking mode of FIG. 2. However, the increase in safety can greatly outweigh the slight decrease in efficiency of the dialysis operation. Thus, in one embodiment, the normal mode proceeds for about 60 seconds, and then the air checking mode of FIG. 2 proceeds for about ½ second after every one minute of normal mode session. FIG. 3 shows how flow through the venous line is blocked when air 60 is detected in line 34 due to an accidental separation of fistula needle 62 from the patient. As stated, an alarm may be sounded to alert the operators of the system, and the patient's life is saved with only a limited loss of blood.

Clamps 48 and 56 are both used to shut off flow from the upstream portion of the venous line 34. Thus, 100 percent of the flow comes through the downstream portion 35 of venous line 34, and no flow comes through upstream venous line portion 37, to increase the reverse flow and to be sure that any air present downstream in the vicinity of connections 52 and 54 is brought rearwardly in flow direction 50a to air sensor 18, as in FIG. 2.

Normally, if no air is detected in the ½ second or so duration of the mode of FIG. 2, the system restarts its normal mode of operation of FIG. 1 for another predetermined time such as 60 seconds. The entire dialysis procedure may continue in this manner, with safe monitoring of the patient, with greater confidence that a catastrophic blood loss can be avoided.

Periodically, if desired, clamp valve 48 may be left open during the air sensing mode, so that negative pressure extends through the entire venous set 34, to check for leaks upstream of clamp valve 48.

As stated, if air is detected as in FIG. 3, the entire system shuts down, and an alarm may be sounded. Thus, a sleeping patient is protected, even if the patient is at home alone, undergoing hemodialysis. The shut-down preferably closes valves 48 and 56, and roller pump 20 stops for a further bloodline closing. An alarm will also be actuated.

Only one of clamps 48 or 56 need to be present to achieve their particular advantage. Clamp 56, is a typical feature found in the dialysis hardware which may be modified in accordance with this invention by the addition of air sensor assembly 17 comprising air sensors 16, 18, and valve assembly 43, which comprises the H-shaped tube construction 42 and bar clamps 46, 48, connected by a connector wire 49 so that the air sensors 16, 18 can signal the conventional clamping system (not shown) that can actuate bar clamps 46, 48. Assemblies 17, 43, and the connecting wire 49 can comprise a part of the tubing set system shown in FIG. 1 that connects to dialyzer 30. The valve actuator may be a conventional device, comprising an added part of the dialyzer hardware. Thus, conventional dialyzer machines may be modified to function in accordance with this invention.

Alternatively, clamp 56, comprising part of the conventional dialyzer hardware, may also be used alone as a control for the system without clamp 48, to close when clamp 46 opens for bubble detection as shown in FIG. 3, for example when the improvement of this application is built into dialysis hardware apparatus as original equipment and not as an add-on device.

The above has been offered for illustrative purposes only, and is not intended to limit the scope of the invention of this application, which is as defined in the claims below.

That which is claimed is:

1. The method of monitoring for leaks or disconnections in an extracorporeal blood circuit comprising a blood pump, an arterial blood flow portion operating at subatmospheric pressure and extending upstream from the pump to a first connection with the patient's vascular system, and a venous blood flow portion extending downstream from the pump to a second connection with the patient's vascular system, which method comprises:
operating said blood pump to circulate blood through said extracorporeal blood circuit; opening a shunt connection between the arterial and venous blood flow portions; sensing the presence of air from any leaks or disconnections within said venous blood flow portion; and taking corrective action if the presence of said air is noted.

2. The method of claim 1 in which said shunt connection is briefly opened and closed on a repeated basis.

3. The method of claim 1 in which said presence of air is sensed by a sensor located to sense for said air near to said second connection.

4. The method of claim 2 in which said shunt connection is opened and closed using only a single unclamping/clamping action.

5. The method of claim 1 in which said venous blood flow portion is clamped, as said shunt connection is opened, at a position to promote blood flow through said shunt connection from the venous blood flow portion downstream of said shunt connection to the arterial blood flow portion operating at subatmospheric pressure, to promote flow reversal in a section of said venous blood flow portion that connects with the patient's vascular system.

6. The method of claim 1 in which said shunt connection is opened for no more than about one second at a time.

7. The method of claim 6 in which said presence of air is sensed by a sensor located to sense for said air near to said second connection.

8. The method of claim 7 in which said venous blood flow portion is clamped, as said shunt connection is opened, at a position to promote blood flow through said shunt connection from the venous blood flow portion to the arterial blood flow portion operating at subatmospheric pressure, to promote said flow reversal in a section of said venous blood flow portion that includes said second connection with the patient's vascular system.

9. The method of claim 8 in which said arterial blood flow portion continues to convey blood through said first connection with the patient's vascular system and to convey said blood away from said patient, when said flow is being reversed in said venous blood flow portion, and also when said flow is normal in said venous blood flow portion.

10. The method of claim 1 in which said arterial blood flow portion continues, by action of said blood pump, to convey blood through said first connection with the patient's vascular system and to convey said blood away from said patient, when said flow is being reversed in said venous blood flow portion, and also when said flow is normal in said venous blood flow portion.

11. The method of claim 1 in which, by action of said blood pump, blood flows from said patient through the first connection with the patient's vascular system into the arterial blood flow portion and away from said patient when said shunt connection is open, and also when said shunt connection is closed but blood is circulating through said extracorporeal blood circuit.

12. The method of claim 1 in which said shunt connection comprises a single flow path.

13. An extracorporeal blood circulating device, which comprises:
a blood pump;
an arterial blood flow tube portion extending upstream from the pump to a first connector for connection with the patient's vascular system;
a venous blood flow tube portion extending downstream from the pump to a second connector for connection with the patient's vascular system;
a shunt connection permitting direct flow between the arterial and venous blood flow tube portions without passing through the blood pump;
a first valve controlling flow through said shunt connection; and
a first valve control unit.

14. The blood circulating device of claim 13, further comprising a second valve to block flow through a portion of said venous blood flow portion which is upstream in normal flow from said shunt connection, said control unit normally causing said second valve to be open when the first valve is closed, and the second valve to be closed when the first valve is open.

15. The blood circulating device of claim 14, in which the presence of air is sensed by a sensor located to sense for said air near to said second connector and to shut down said system when air is sensed.

16. The blood circulating device of claim 14, in which said first and second valves use only a single unclamping/clamping action.

17. The blood circulating device of claim 14, in which said shunt connection comprises a single flow path.

18. The blood circulating device of claim 17, in which said first and second valves use only a single unclamping/clamping action.

19. The blood circulating device of claim 18, further comprising an air sensor located near to said second connector

20. The blood circulating device of claim 13, in which the presence of air is sensed by a sensor located to sense for said air near to said second connector and to shut down said system when air is sensed.
